(19) United States
(12) Patent Application Publication
(10) Pub. No.: US 2024/0389994 A1
(43) Pub. Date: Nov. 28, 2024

(54) IMPROVED BONE ANCHOR AND BONE ANCHOR INSERTION TOOL

(71) Applicant: PBC INNOVATIONS LIMITED, Shannon, Co. Clare (IE)

(72) Inventors: Robert A. FLAVIN, Shannon (IE); Ivana IVANKOVIC, Shannon (IE)

(21) Appl. No.: 18/692,522

(22) PCT Filed: Sep. 16, 2022
(86) PCT No.: PCT/EP2022/075730

§ 371 Date: Mar. 15, 2024

Related U.S. Application Data
(60) Provisional application No. 63/245,183, filed on Sep. 16, 2021.

(51) Int. Cl. A61B 17/04

(52) U.S. Cl. CPC A61B 17/0401; A61B 2017/0409; A61B 2017/044

(57) ABSTRACT

A bone anchor for rotator cuff repair, comprising a solid cylinder extending in a proximal to distal direction and defining a longitudinal axis (L-L); a thread on the solid cylinder encircling the solid cylinder at least twice; a connection, at the proximal end of the solid cylinder, for connecting to a tool to drive the thread into bone; an unthreaded endpiece, extending from the distal end of the solid cylinder, having a distal end; and a tunnel across the unthreaded endpiece, wherein the thread is configured to define at least two straight longitudinal passageways from the distal end of the solid cylinder to the proximal end of the solid cylinder.

### CROSS-REFERENCE TO RELATED APPLICATIONS

This application is a U.S. National Stage of International Patent Application No. PCT/EP2022/075730 filed Sep. 16, 2022 which claims priority to U.S. Provisional Application No. 63/245,183 filed Sep. 16, 2021, both of which are incorporated herein as if reproduced in their entirety.

### TECHNICAL FIELD

The present application relates to a bone anchor for reattaching tendons to bones. The bone anchor is optimized for use with bone adhesives and is of particular use in surgery to repair rotator cuff injuries.

### BACKGROUND

Tendons are fibrous connective tissue which attach muscles to structures such as bones. Tendons enable muscles to pull on structures and manipulate joints. If tendons get damaged, surgery may be required to restore a required level of operation of the joint or to avoid pain when the joint is manipulated.

A human rotator cuff is an example of a site that often requires surgery to repair significant tendon damage. In a rotator cuff, a collection of muscles and tendons connect the scapula (shoulder blade) to the humerus (upper arm bone). The tendons and muscles essentially hold a shoulder in place whilst permitting the various motions of the associated arm. Injuries to rotator cuffs can arise from trauma or degeneration and are one of the most common types of tendon injury that is treated. As humans age, the likelihood of requiring surgical treatment increases, while their bone quality decreases.

Known surgical treatment techniques for rotator cuff repair include bone anchor implantation and suturing. Bone anchor implantation essentially affixes a bone anchor to a bone. A separated tendon can then be sutured to the bone via the bone anchor. However, the implantation failure rate is high—the bone anchor pulls out of the bone in over 50% of cases, often requiring a second surgery.

The risk of implant failure is even higher if the surgery requires that an implant is placed in bone which is low-quality. Low-quality bone is bone that is not well-suited for implantation. There are many reasons bone might be low-quality such as osteoporosis or other disease, trauma or other disruption, or the target site comprising cancellous/trabecular (spongy) bone as opposed to cortical (hard) bone.

A bone adhesive may also be used to adhere the bone anchor to the bone. Bone adhesives have also been developed for use in low-quality bone. However, it is difficult to create a bone adhesive that bonds well to bone anchors and low-quality bone.

Therefore, even with bone adhesives, bone anchor implantation has a high failure rate and is challenging to perform at low-quality bone sites. Consequently, there is a need for an improved bone anchor that will address at least some of these problems.

### SUMMARY

Bone anchors are described that are particularly suitable for use in rotator cuff repair surgery. The bone anchors according to the exemplary arrangement of the specification have been configured to work with bone adhesives and are well-suited for use in low-quality bone. The bone anchors have a low failure rate relative to known bone anchors, and their secure hold also decrease the likelihood of other adverse effects.

The bone anchors have been configured with a proximal solid cylinder part for screwing into bone, and a distal unthreaded endpiece with a rounded end to provide a surface that is well-suited to bonding with bone adhesives. As the endpiece is free of protruding structures it is well suited to low-quality bone and allows repositioning with little damage to local bone.

The bone anchors have at least two straight longitudinal passageways on the outside, and there is a tunnel through the unthreaded endpiece of the bone anchor. A strand of suture may be placed through the tunnel and longitudinally along the two passageways. The passageways guide the strand of suture through bone-engaging elements on the outside of the bone anchor. The use of the tunnel allows minimally invasive arthroscopic surgery, as it allows for minimalistic knot tying techniques and knot locking.

Additionally, by passing the suture through a tunnel in the endpiece, more of the proximal end of the bone anchor is available to engage with bone. The proximate end of the bone anchor is typically located in the best quality of bone. Therefore, the bone anchor ensures a secure fit even if the distal end of the bone anchor is in low-quality bone.

When the bone anchor is fixed in the bone, the bone and/or bone adhesive helps lock the strand of suture to the bone anchor. The bone anchor then has two strands of suture emerging, and each strand emerges from a different location from a respective passageway. This configuration allows surgical techniques to be used based on two strands of sutures emerging from two different locations.

Further, relative to using a single or double strand of suture emerging from one location, the stress is spread out which helps reduce the risk of the bone anchor or suture failing. As the strands of suture emerge from different locations, when the strands are joined around a tendon and tension is applied, the strands do not pinch together. This helps avoid the suture strands damaging the tendon or pulling through the tendon.

The bone anchor may also have a bone engaging wall that has an asymmetric profile, which is less steep on the proximal side than on the distal side. Therefore, a surgeon can easily pull, or twist, out the anchor and reposition the bone anchor if necessary. Due to the asymmetric profile, the twisted retraction of the anchor is likely to cause little damage to the bone and is less likely to damage the thread path that the threads make in the local bone during insertion.

The scope is in accordance with the appended claims. According to the disclosure there is provided a bone anchor, an insertion tool, a sleeve, and a bone anchor insertion kit as defined in the independent claims. Further optional features are provided in the dependent claims.

### BRIEF DESCRIPTION OF THE DRAWINGS

Embodiments of the present disclosure will now be described, by way of example, with reference to the accompanying drawings, in which:

FIG. 1a shows schematically a rotator cuff;

FIG. 1b shows schematically the rotator cuff of FIG. 1a with a damaged tendon;

FIG. 1c shows schematically the damaged tendon of FIG. 1b when repaired using a known surgical process;

FIGS. 2a to 2c shows schematically the steps to repair the damaged tendon of FIG. 1b with a bone anchor according to an exemplary arrangement of the present specification;

FIG. 3 shows schematically a bone anchor according to some embodiments of the present specification;

FIG. 4 shows a bone anchor according to some exemplary arrangement of the present specification;

FIG. 5 shows an end-on view of the bone anchor shown in FIG. 4, looking in the distal direction;

FIGS. 6a to 6d shows a bone anchor according to some embodiments of the present disclosure;

FIGS. 7a to 7c show a bone anchor according to an exemplary arrangement of the present specification;

FIG. 8a shows a cross-sectional view of the bone anchor insertion tool of connected to a bone anchor of the exemplary arrangements of the specification at the distal end; and FIG. 8b shows cross-sectional view of an alternative bone anchor insertion tool having a port for attachment of a syringe;

FIG. 9 shows the connection between a bone anchor insertion tool according to an exemplary arrangement of the specification and a bone anchor according to an exemplary arrangement of the specification in this case for example FIG. 6 above; and

FIG. 10a shows a cross-sectional close up view of the handle portion when in a first closed position; FIG. 10b shows a partially cut away perspective view of the of the proximal handle portion in the open position; FIG. 10c shows the handle in the open position in a cross-sectional view.

### DETAILED DESCRIPTION

A rotator cuff can be considered as the mechanical linkage between the scapula to the humerus and comprises a collection of muscles and tendons. As explained above, sometimes a tendon in this collection gets worn or damaged and needs surgery to support or reconnect the tendon to the bone.

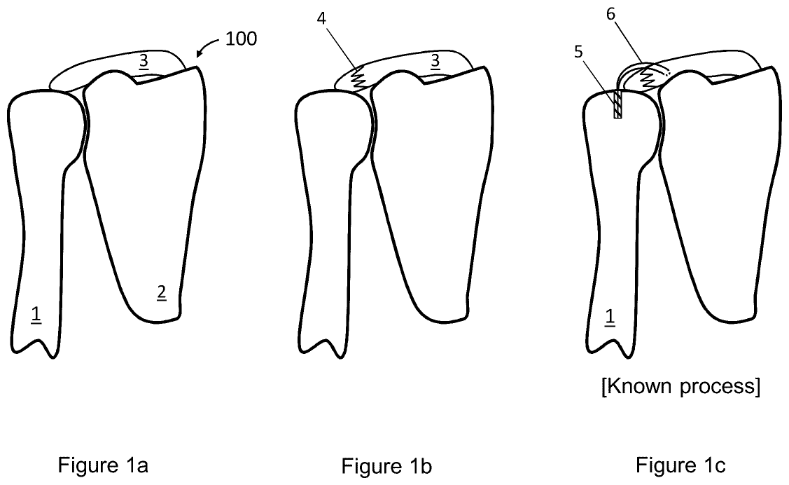

FIG. 1a show a schematic image of the relevant anatomy. The humerus 1 is connected to the scapula 2 via a curved sliding interface. A tendon 3 connects the humerus 1 to a muscle, for example the supraspinatus muscle. Contraction of the muscle will pull the tendon 3 to manipulate the humerus 1 relative to the scapula 2.

FIG. 1b shows a patient with a damaged rotator cuff. Specifically, it shows a tear 4 in the tendon 3. Such a tear 4 can reduce mobility of the joint and cause the patient pain. In severe cases, a patient may find it almost impossible to move the affected arm.

The patient could be treated with a known surgery method to repair or support the tear, in the manner shown in FIG. 1c. The method involves surgically implanting a bone anchor 5 into the humerus 1. The bone anchor 5 includes a feature around which a strand of suture 6 can be looped. The looped suture 6 can then be secured to the tendon beyond the tear 4 and tightened, securing the tendon 3, and reducing the pressure on the tear 4.

Known bone anchor designs operate best in strong, or high-quality, bone. Known designs do not operate well in low-quality bone, in which they are often implanted, as low-quality bone is common for many reasons. For example, Osteoporosis, which will affect 1 in 4 women and 1 in 5 men globally, is a bone disease causing lower bone density and therefore much weaker bones that are prone to fracture. Implants, such as bone anchors, are much more likely to fail, e.g., pull out if they are implanted in osteoporotic bone.

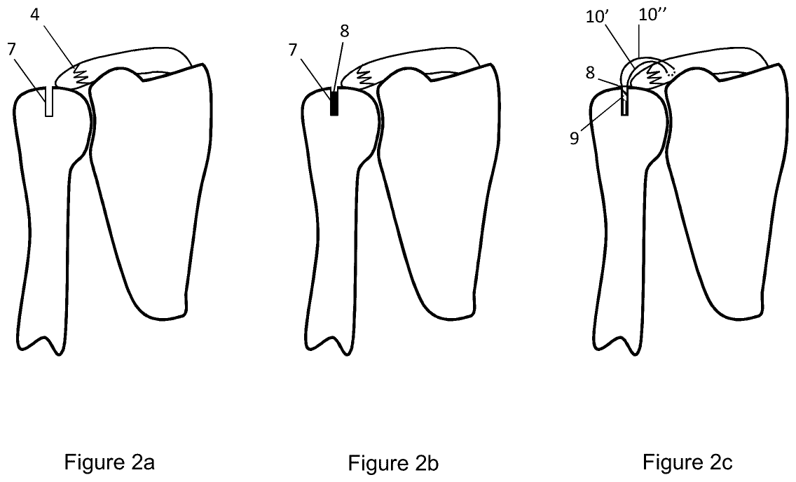

A new bone anchor has been designed that can perform well in all bone, including low-quality bone. The process of use is shown in FIGS. 2a to 2c.

Considering a tendon tear 4 like that described above in FIG. 1b, the first step shown in FIG. 2a is to form a hole 7 in the humerus 1. This might be achieved by surgically drilling or any other known method.

As shown in FIG. 2b, this hole 7 is then filled with a bone adhesive 8 that bonds strongly to bone. Many known adhesives may be used such as other calcium phosphates or synthetic bone cement. Some of these adhesives may help achieve better augmentation of the implanted bone anchor in low-quality bone and/or may promote bone growth.

As shown in FIG. 2c, the bone anchor 9 with an inserted strand of suture is then inserted into the hole with the adhesive 8. Once the bone anchor 9 is secure, two strands 10′, 10″ of suture are available to attach. Each strand of suture can thus be used to form a connection between the humerus and the tendon. Alternatively, the strands can secure the tendon by passing the strands through the tendon and connecting the strands together.

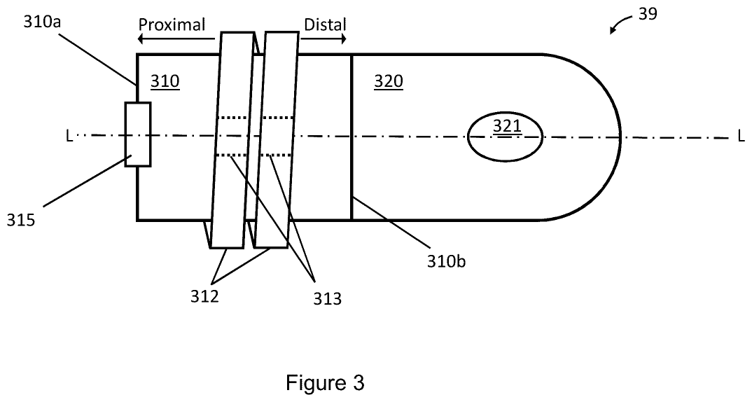

Additional details of some embodiments are shown in FIG. 3. In these, the bone anchor 39 comprises a solid cylinder 310 that defines a longitudinal axis L-L. The solid cylinder 310 extends in a proximal to distal direction. The proximal end 310a of the solid cylinder 310 is the end that is closest to the surgeon in normal use, i.e., the end that is closest to the top of the hole in the bone after the bone anchor 39 has been implanted.

The solid cylinder 310 has no tunnels, passageways, voids, or other spaces in it. Therefore, there are no spaces that are mechanically isolated from the body, in which infection might take hold.

On the solid cylinder 310, there is at least one wall 312 that encircles the solid cylinder 310. In other words, the wall 312 extends around the circumference of the solid cylinder. The wall 312 forms a helical pattern around the solid cylinder 310 encircling the solid cylinder 310 approximately twice (720 degrees). The wall 312 is configured to engage with a bone and secure the bone anchor 39 into the bone when it is implanted.

In other embodiments, the wall may by circularly symmetric and/or there may also be multiple walls. Each wall may also be continuous; or formed of several discrete and/or separate parts. Any variation of wall will be usable provided it can firmly secure the bone anchor 39 into bone.

The wall 312 is configured to define a straight passageway 313 in the longitudinal direction, that is suitable for passing a strand of suture through the wall 312.

An unthreaded endpiece 320 distally extends from the distal end 310b of the solid cylinder 310. The endpiece 320 is designed to provide a portion of the bone anchor 39 that is well suited for bonding to a bone adhesive. The endpiece 320 is substantially smooth and defines a cylindrically symmetric shape comprising a proximal cylinder and a rounded end extending from the distal end of the proximal cylinder. The proximal cylinder is the same radius as, and concentric with, the solid cylindric 310.

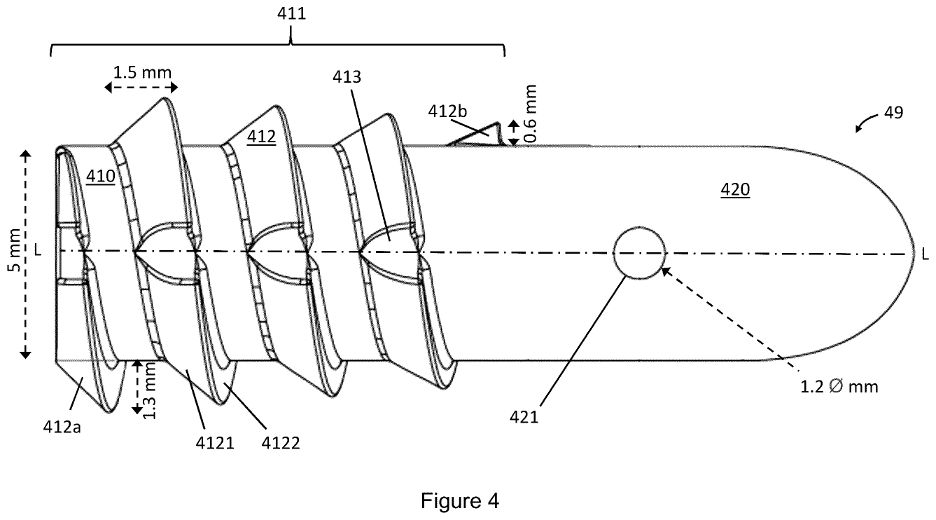

The radius of the rounded end is between 1 and 5 mm and can be set for each embodiment according to requirements. In embodiments having relatively small radii, the cylindrically symmetric shape is a substantially a cylinder with a rounded distal edge. In other embodiments, such as that shown in FIG. 4, the cylindrically symmetric shape is bullet-shaped having a rounded end with a radius that is at least as big as the radius of the proximal cylinder.

The endpiece 320 surface is unthreaded and unobstructed by protruding features such as threads. Therefore, bone adhesive 8 can very effectively bond to the endpiece 320.

Additionally, the shape of the endpiece 320 is well-suited for use in low-quality bone. This is because pressing protrusions, e.g., walls or threads, into low-quality bone typically causes damages and does not result in much support. In addition to providing an effective surface for bonding, the rounded end of the endpiece 320 can help to compress the bone into which it is pressed which can assist in the bonding process between the bone anchor 39, bone adhesive, and low-quality bone. The lack of protrusions also means that the endpiece 320 will not cause any damage when it is removed, which is advantageous in cases when a surgeon needs to reposition the bone anchor 39.

The surface of the endpiece 320 may be further treated to improve the bonding by surface treatment. For example, the endpiece 320 may be etched; textured; plasma treated; anodized, preferably with type II (often sulfuric acid based) anodizing; polished; cleaned; or coated with an intermediate thin film. This treatment may be limited to the endpiece 320 in some cases. In other cases, for ease of production, other parts or the whole bone anchor 39 may be treated equivalently.

The material of the bone anchor 39, at least for the endpiece 320, may be selected to improve the bonding. The selection of the material will take into account other factors such as mechanical properties, biocompatibility, and machinability. Typically, the selected material will be a medical grade surgical implant material. For example, whole bone anchors or part thereof may be formed from, or comprise, at least one of Titanium, PEEK, stainless steel, cobalt chrome. For some embodiments, the bone anchor 39 will be formed from multiple materials to optimise the material properties of individual parts.

In one preferred example, the whole bone anchor 39 is formed from Titanium Grade 5 (Ti6Al4V) because this material provides good mechanical and biological properties, and an endpiece 320 in this material bonds well with bone adhesive.

At the proximal end 310a of the solid cylinder 310, there is a connection 315 for connecting to a driving tool to drive the bone anchor 39 into bone. The connection 315 allows a surgeon to use a driving tool to insert and/or retract the bone anchor 39. Multiple forms of connections are possible including machined receptables or protrusions for coupling to matching tools e.g., hex slots and hex drivers; features to facilitate gripping such as knurling; and magnetic coupling. Any feature that allows a surgeon to forcibly manipulate the bone anchor 39 from the proximal end can be used as a connection.

The endpiece 320 further comprises a tunnel 321. The tunnel 321 is straight and passes through the endpiece 320 to the opposite side. There are therefore two opposing openings to the tunnel 321 in the endpiece 320. A strand of suture can be inserted through the tunnel 321. In contrast to some known bone anchors, tunnel 321 does not extend longitudinally inside the bone anchor 39. Instead, the tunnel 321 extends perpendicularly to the longitudinal axis and the longitudinal axis instead passes perpendicularly through the centre of the tunnel 321. As there is no long longitudinal tunnel, there is little space inside the bone anchor 39 in which infection might occur. This is a significant advantage, as it is very difficult to treat infections if they occur in areas that are mechanically isolated from the body.

In other embodiments, there can be multiple tunnels. In some embodiments, the one or more tunnels can be located at different places in the endpiece 320 and have different orientations, shapes, or sizes.

In FIG. 3, the solid cylinder 310, and endpiece 320 extend for roughly half the longitudinal length of the bone anchor 39 each. This however is merely an example and other embodiments may have other portions or other parts depending on the design requirement for a particular surgical scenario. In particular, the solid cylinder may extend between 0.1 and 0.75 times the overall length of the bone anchor.

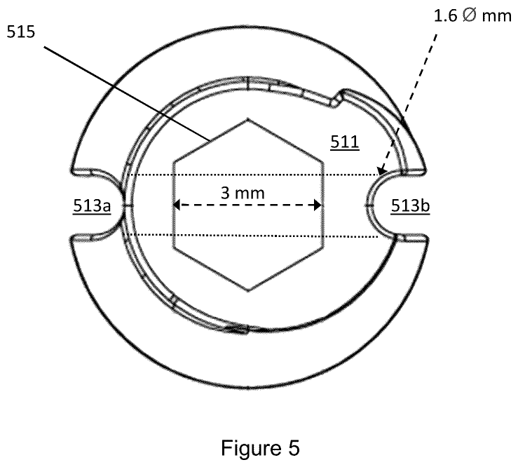

FIG. 4 provides further details of some embodiments of the disclosure. It should be noted that the dimensions in FIGS. 4 and 5, shown with dotted lines, are only preferred dimensions—other embodiments may use other dimensions. Analysis of stress has however shown that the preferred dimensions are the optimal dimensions for bone anchors for use in rotator cuff repair surgery.

In the embodiments shown in FIG. 4, the endpiece 420 has a circular tunnel 421 that is perpendicular to, and centred on, the longitudinal axis L-L. The circular tunnel 421 provides a path through the endpiece 420 through which a strand of suture can be moved. The circular tunnel 421 is located approximately in the longitudinal middle of the endpiece 420.

The screw-part 411 of the bone anchor 49 comprises a solid cylinder 410 and an encircling helical wall forming a thread 412 that encircles the solid cylinder 410 approximately three and a half times (1260 degrees). The thread 412 is dimensioned to provide secure connection to the bone into which the bone anchor 49 is placed. Some embodiments use standard threads. However, the profile of the thread 412 can be optimized as shown in FIG. 4. The optimized thread is specifically designed to work in low-quality bone

The optimization includes variation of the protrusion of the thread 412 along its length. In FIG. 4, the thread protrusion, or depth, as measured from the solid cylinder 410, decreases from the (proximal) start of the thread 412a to the (distal) end of the thread 412b. In FIG. 4, the start of the thread 412a, at the proximal end of the anchor 39, protrudes 1.3 mm; and the end of the thread 412b, approximately mid-way along the bone anchor 49, protrudes 0.6 mm. The variation in the protrusion of the thread 412 helps account for the variation in bone quality (when implanted, the proximal end of the bone anchor 49 will be closest to the exterior surface of the bone where the quality is likely to be highest). The longitudinal length of the thread 412 remains constant, at approximately 1.5 mm, along the thread.

The thread 412 also has an asymmetric profile, or cross-section. The asymmetric profile comprises two profiles: a proximal profile 4121 and a distal profile 4122. Such an asymmetric profile is known as a reverse cut (downward-facing) screw. The two profiles have different angles from the longitudinal axis L-L. The proximal profile 4121, which is preferably less than 75 degrees and more preferably 45 degrees or less, has a slope that is less inclined than the slope of the distal profile 4122. To achieve a variation in thread protrusion, the incline of the proximal profile 4121 decreases from the start of the thread 412a to the end of the thread 412b.

An effect of the asymmetric thread profile is that if the bone anchor 49 needs to be repositioned during implantation, the asymmetric thread profile makes it easier for a surgeon to twist out the implant without damaging nearby i.e. local bone.

To explain this effect, the bone anchor 49 is inserted into the bone by applying a small force in the distal direction and a twisting insertion motion in a clockwise direction (looking from the proximal end around the longitudinal axis L-L). During insertion, the thread 412 cuts or deforms the bone that it engages with to form a thread path in the bone. The engagement of the thread 412 with the formed thread path secures the bone anchor 49 in the bone. The process is like to screwing a wood screw into wood.

If necessary, application of a twisting retraction force applied in an anti-clockwise direction (looking from the proximal end around the longitudinal axis L-L) causes the thread 412 to engage with the thread path retracting the bone anchor 49 out of the bone. In known bone anchors, there is a risk that the thread 412 will slip out of the thread path, spin in the bone hole, and damage the local bone. In some cases, a damaged thread path results in the bone anchor simply slipping and/or spinning within the bone when a twisting retraction force is applied. In such cases it can be very difficult to retract the bone anchor without causing further damage to local bone.

The asymmetric profile helps reduce this risk. The relatively steep distal profile 4122 ensures the thread 412 and thread path make firm contact reducing the risk of the thread 412 slipping past the thread path. The relatively flat proximal profile 4121 also makes it easier to retract the bone anchor 49 by pulling in a longitudinal direction, if necessary.

Additionally, the less-inclined proximal profile 4121 provides a relatively flat area that presses against the bone, which assists the adhesive effect of the bone adhesive. Consequently, the bone anchor 49 is well optimized for use with the bone adhesive.

In other embodiments, depending on the considered surgical scenario, the variation of the thread might take other forms. In particular, the thread 412 can also be optimized by variation along the thread of one or more of: the longitudinal length, protrusion, or shape of the thread profile.

The thread 412 is also formed to define two straight passageways 413 through the thread, in the longitudinal direction L-L. The passageway 413 traverses the walls of the thread 412 in several locations. The passageway 413 permits a strand of suture to run longitudinally past the screw-part 411 without requiring an internal longitudinal tunnel. In this way, there is no long longitudinal tunnels that are a potential infection risk in the bone anchor 49.

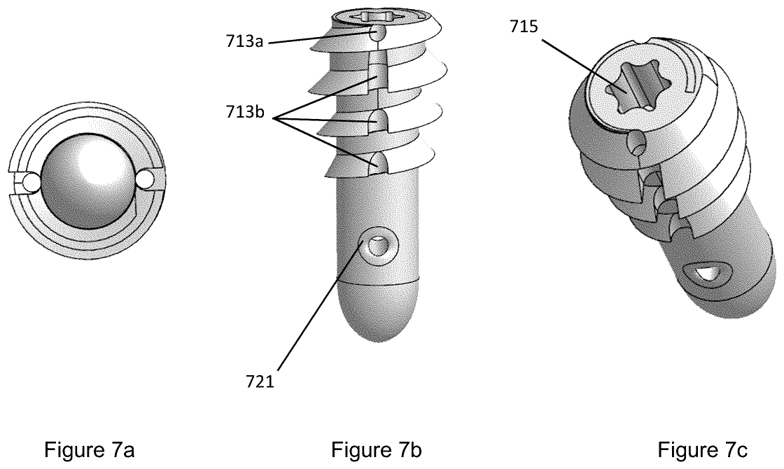

In FIG. 4, the passageway 413 through the thread 412 is open, i.e., it is formed from cavities in the walls of the thread 412. In other embodiments, the passageway may be closed, i.e., formed from several tunnels that are drilled in the walls of the thread. In some embodiments, the passageway is formed from cavities and tunnels. In the embodiment shown in FIGS. 7a to 7c, each passageway is formed so the most proximal crossing of the thread is a tunnel 713a and the other crossing of the thread are cavities 713b. This allows the proximal end of the thread to be larger and/or to have an undisrupted outer edge, which improves grip. Even in these embodiments with closed-passageways, an infection control advantage remains as the depth of tunnel in each thread protrusion is small.

FIG. 5 shows an end-on view of the bone anchor 49, looking in the distal direction at the proximal end 511. In the centre is the connection 515 which, in this case, is a 3 mm hex socket for receiving a matching hex driver. A hex driver has the advantage that, some alignment is provided with the hex driver making it easier for the surgeon to drive in the anchor 49. In other embodiments, other known forms of connections may be used. In particular, a 6-point star socket 715 may be used as shown in FIG. 7c. This is driven by a corresponding 6-point star driver. Such connections also assist in aligning and also reduce the risk of damage to the socket or driver even if a lot of torque is applied to the anchor. The 6-point star socket and driver may be of the Torx® type.

From this end-on view, the two longitudinal passageways 513a and 513b on the opposite side of the anchor 49 can be seen. The tunnel 421 through endpiece 420 (see FIG. 4) is also indicated by dotted lines. A strand of suture can thus be passed down the first passageway 513a, through the tunnel 421, and back up the second passageway 513b; or in the other direction. The passageways in this embodiment have a cross-section profile of a slot with radius of approximately 0.5 mm but any other profile may be used provided it can, at least partly, contain a strand of suture.

The number of longitudinal passageways and their location can be freely adjusted for other embodiments according to surgical need: how many strands of suture are needed and/or how firmly they need to be attached. Similarly, some embodiments, may have multiple tunnels through endpiece 420.

The bone anchor, passageways, and tunnel are formed to avoid sharp edges, e.g., smoothing, using fillets, or polishing edges afterwards. An example of this is shown in the smooth tunnel 715 in FIG. 7b. The threads may also be smoothed into the body of the anchor. This helps limit corners and cavities for infection, and reduces the likelihood of damage to a strand of suture located in or near the various features.

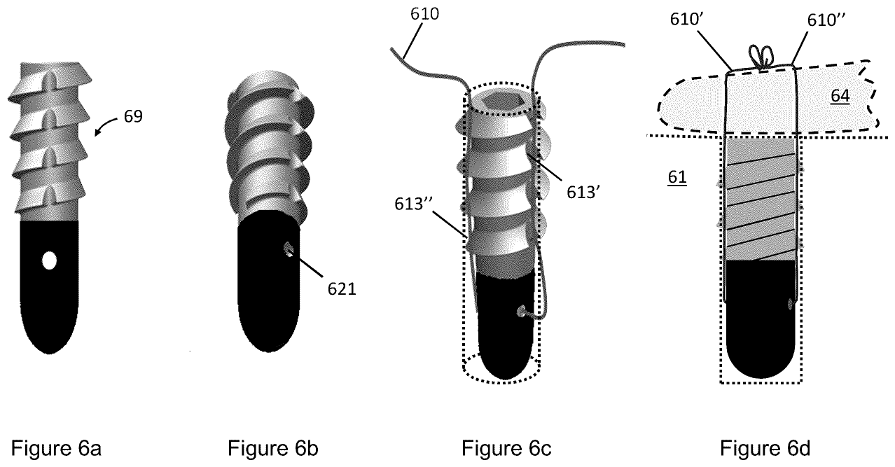

FIGS. 6a and 6b show two rendered images of the bone anchor 69 shown in FIGS. 4 and 5. FIG. 6c shows a schematic of the bone anchor 69 when it has been implanted into a hole in bone 61 filled with bone adhesive. The bone adhesive is not shown for clarity. FIG. 6d shows how the implanted bone anchor 69 can be used to attach a tendon 64.

Due to the straight longitudinal passageways past the thread and the tunnel 621 through the adhesive-securing part, the suture 610 can be placed through the bone anchor 69 before it is screwed or tapped into the hole. Due to the straight longitudinal passageways, as the bone anchor 69 is inserted, the bone adhesive can flow out the passageways avoiding causing excess bone pressure and locating overflowing bone adhesive at the proximal end of the bone anchor 69. The overflow help ensure that the sutures in the passageways are in good contact with the flowing bone adhesive. The overflowing bone adhesive is sandwiched between the tendon 64 and the bone 61, or between the tendon 64 and bone anchor 69. The overflowing bone adhesive is compressed against the tendon 640 by the strand of suture 610 when it is tied.

This compression from the suture 610 helps any overflown bone adhesive bond with the tendon 64. As a result, the implanted bone anchor 69 and tendon 64 are securely attached.

The piece of suture in each passageway is partly held by the pressure of the bone anchor 69 against the bone and/or the bone adhesive. Each piece of suture 610′, 610″ in its respective passageway can therefore provide a holding force.

Consequently, surgery based on using two sutures can be performed even though only one bone anchor 69 has been used. This helps reduce the risk of suture failure and makes the surgery less invasive (as multiple bone anchors are not needed).

Additionally, because the strands of suture 610′, 610″ emerge from the bone anchor 69 at opposite sides, the stress from the suture load is better spread out across the bone anchor 69 and bone 61. This reduces the risk of suture failure and/or bone anchor failure.

FIGS. 6c and 6d shows the bone anchor 69 after it has been implanted into a bone 61. A strand of suture 610 is passed through the tunnel 621 in the endpiece. After the bone anchor 69 and inserted suture has been implanted, two strands 610′,610″ emerge from respective longitudinal passageways 613′, 613″. Each strand of suture emerges at a different location. The strands can then be passed through the tendon 64 and secured together to form a connection between the tendon 64 and the bone 61 via the suture 610 and bone anchor 69. The sandwiched bone adhesive (not shown) will be pressed into good contact by the sutured connection.

In known bone anchors (such as shown in FIG. 1c), strands of suture emerge from the bone anchors alongside each other. As the strands emerge together, they tend to pull or pinch together when tension is applied. This increases the likelihood of the strands cutting through or slipping through a tendon.

In the present disclosure, the emerging strands 610′, 611″ are separated when they emerge from the bone anchor 69. The separation is approximately 5 mm in the embodiment shown in FIGS. 6a to 6d. The separation helps prevent the strands pinching together when tension is applied. Consequently, the bridge that is formed across the top of tendon 64 by strands 610′ and 610″ is more stable and the tendon 64 is more firmly secured to the bone 61. In other words, by having the separation of the emerging strands 610′,610″, it is easier to capture the tendon with the strands.

According to a further aspect, the specification provides exemplary arrangements of a bone anchor insertion tool 1000, 1000′ configured for use for inserting a bone anchor according to the arrangements of the specification.

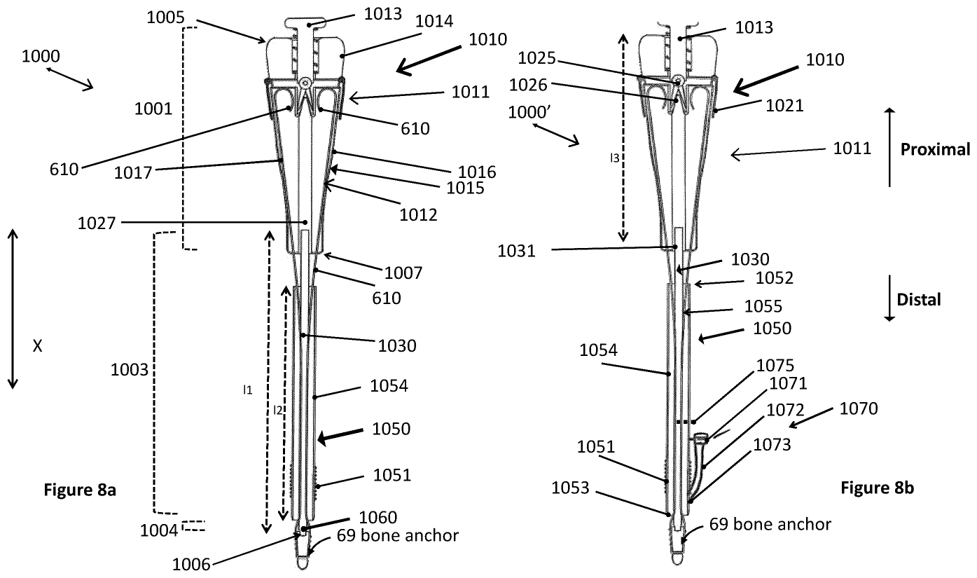

FIGS. 8a and 8b shows a cross-sectional view of bone anchor insertion tools 1000, 1000′ connected to a bone anchor 69 at the distal end. The bone anchor insertion tools 1000, 1000′ are similar, and a common description is provided below with reference to the drawings, the same reference numbers have been used. The bone anchor insertion tool 1000′ further includes a syringe port and stop feature 1070, described further below.

The bone anchor insertion tool 1000, 1000′ has a longitudinal form extending from a proximal end 1005 to a distal end 1006. The bone anchor insertion tool 1000, 1000′ generally comprises three portions a proximal handle portion 1001 and an elongated shaft portion 1003, wherein a shaft 1030 extends distally from the handle portion to a distal bone anchor engagement portion 1004 for engagement with a bone anchor, for example, bone anchor 69. The bone anchor insertion tool 1000 is arranged about a central vertical axis, extending in the longitudinal direction illustrated in the drawings as the X-direction.

The proximal handle portion 1001 comprises a handle assembly 1010 having a proximal end 1005 and a distal end 1007. The handle assembly 1010 has a generally tapered cylindrical form or generally conical form that tapers from the proximal end to the distal end. The handle assembly 1010 has length 13 which may be for example of the order of 6.5 cm, in an exemplary arrangement the diameter of the handle may be of the order of 3.5 cm at widest upper proximal portion 1005 tapering to around 1.5 cm at the distal end 1007 of the handle assembly.

The handle assembly 1010 comprises a body 1011 having an upper cap portion 1014 and a lower side wall portion 1015 that defines an internal housing 1012, for housing the actuating means for example a hinge and lever system 1025, needles and sutures. The side wall 1015 is a peripheral side wall and comprises an external surface 1016 and an internal surface 1017. The external surface 1016 defines a user contact surface 1016 of the handle that is gripped by a user for operation of the bone anchor insertion tool 1000. The handle assembly 1010 accordingly defines a first user actuation means that allows for the user to affect a driving of the bone anchor into the bone. The handle assembly further comprises a second user actuation means or actuator 1013. The actuator 1013 in the exemplary arrangement comprises a push button 1013 operable to release the sutures and needles from the handle to use for the tendon repair. The bone anchor insertion tool 1000, and in particular the handle assembly 1010 and push button 1013, are advantageously configured to allow for a single-hand operation to release the device. This is of benefit to the user taking account of the constraints of a bone anchor implanting surgical procedure.

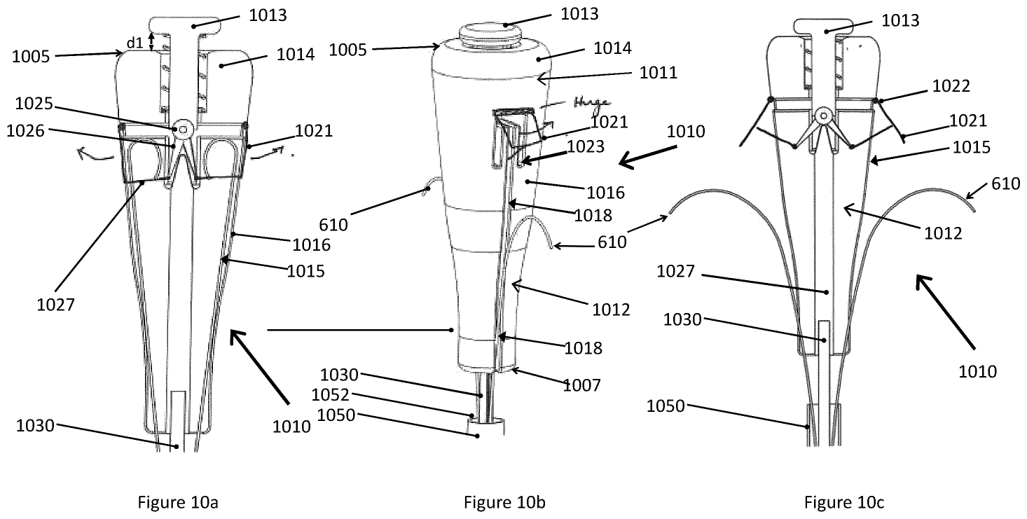

The handle assembly 1010 may be gripped at the external contact surface 1016 during a procedure and forces applied via the handle to drive the bone anchor as required for example by rotation or turning of the handle. The forces are transmittable via the shaft 1030. The push button actuator 1013 may be pressed or actuated to provide the release of the sutures and needles from the handle through the horizontal hinged door flaps. These features are described further below with reference to FIG. 10.

The handle assembly 1010 comprises a lumen 1027. Shaft 1030 is arranged extending distally from the handle portion 1010 in the direction of the bone anchor.

The shaft 1030 extends from a handle connection portion 1031, at which it is connected to the handle, to a bone anchor connection head 1060. The shaft 1030 as shown in exemplary FIG. 8a, is arranged in use to extend through a sleeve 1050. The shaft 1030 is moveable in the longitudinal direction (+X/−X) relative to the sleeve 1050. The shaft can be moved distally in the direction of the bone or retracted proximally in the direction of the handle, relative to the sleeve. As shown, the suture 610 is arranged extending from the bone anchor 69 to the handle assembly 1010 through the sleeve 1050. The shaft 1030 has a length 11, the sleeve has a length 12 shorter than that of the shaft. The length 11 of the shaft is selected taking account of the particular parameters of the target surgical area, the depth requirements, the bone that is the subject of the surgery and can be varied as required.

Bone anchors according to the specification have a connection socket 615 for receiving the distal head 1060 of the insertion tool.

Referring to FIG. 8B bone anchor insertion tool 1000′ comprises sleeve 1050 and further comprises a fluid input arrangement 1070, which is essentially provided as a set of additional features to the insertion tool 1000.

The sleeve 1050 comprises a proximal end 1052, a distal end 1053. The fluid input 1070 is located near to the distal end 1053. The sleeve 1050 comprises a peripheral wall 1054 of generally cylindrical form. The sleeve 1050 defines and internal channel 1055 or lumen though which the shaft 1030 and sutures 610 passes. A portion 1051 of the external surface of the wall 1054 is threaded.

The fluid input 1070 comprises a syringe dock 1071 for receiving a syringe, tubing 1072, and a fluid entry port 1073. The fluid entry port 1073 is formed as an aperture that extends through the wall 1054 in the sleeve 1050. The tubing 1072 provides a fluid channel between the syringe and the sleeve 1050 to allow for the introduction of fluid into the interior channel 1055 of the sleeve. The distal end of the sleeve 1053 is configured for coupling at or near to a proximal end 611 of the bone anchor to allow for the fluid to be delivered via the sleeve to the bone anchor 69.

The insertion tool 1000′ further comprises a stop 1075 or lock 1075 located at the sleeve 1050 and that is operable to allow for the shaft 1030 to be retained proximally of the fluid entry port 1073, if a fluid is to be introduced to the sleeve, and to allow for release of the shaft 1030 when the fluid has been introduced. The stop is movable between a closed position and an open position when the shaft is released to move relative to the sleeve. The stop 1075 and shaft 1030 may include corresponding mating features. In an alternative arrangement the stop may be locatable in the sleeve to prevent advancement of the shaft.

While the sleeve 1050 comprising a fluid input 1070 and stop 1075 is described above with reference to insertion tool 1000′, it will be appreciated that according to a further aspect the specification provides a sleeve 1050 including a fluid input 1070 and stop 1075 useable with various type of insertion tools and bone anchors or other implants. The arrangement may be used to selectively deliver a fluid to a surgical site. The fluid may include for example, a bone cement, surgical adhesive, or other suitable fluids as may be required during surgery.

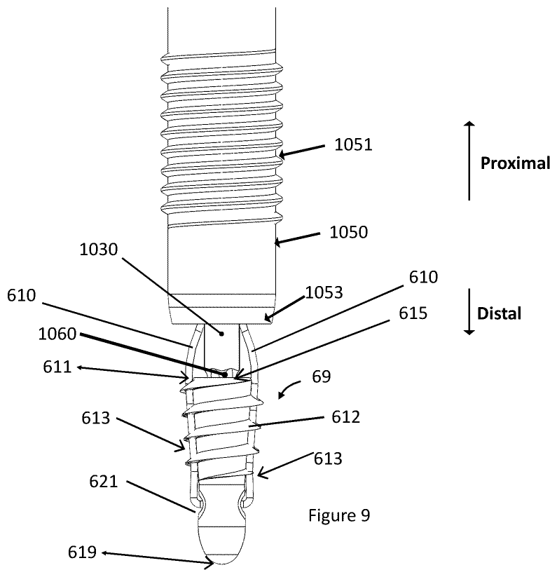

Referring to FIG. 9, the connection between a bone anchor insertion tool 1000 or 1000′ according to an arrangement of the specification and a bone anchor according to an arrangement of the specification is described in further detail.

The bone anchor 69 shown as an example comprises a proximal connection socket 615, at a proximal end 611, a distal end 619, a thread 612 and tunnel 621. Suture 610 is shown extending from the bone anchor which it is threaded through the tunnel in the proximal direction into the sleeve and co-located with the shaft 1030. The bone anchor 69 comprises externally located longitudinal passageways 613 in which the suture 610, in this case both strands of the suture 610 are locatable. The suture 610 also passes through the tunnel 621. After the bone anchor 69 and inserted suture 610 has been implanted, two strands of the suture 610 emerge from respective longitudinal passageways 613 extending in the proximal direction. Each strand of suture emerges at a different location and extends from the bone anchor into the sleeve 1050 and through the sleeve in directions generally parallel to the shaft 1030 to needles 1027 located in the handle assembly 1010.

As noted with reference to FIG. 8b, with reference to insertion tool 1000′ the sleeve 1050 may be located at or near the bone anchor to allow for delivery of a fluid to the bone anchor directly via the sleeve.

The head 1060 of the shaft 1030 is engaged with the connection socket 615 of the bone anchor. The head 1060 and the connection sock 615 comprise corresponding mating features. As the insertion tool is operated by the user forces are transmitted via the insertion tool to the head 1060 which is engaged with the bone anchor 69 to move the bone anchor, as required.

While in the exemplary arrangement bone anchor 69 is shown, it is appreciated the insertion tool is configured for use with bone anchors of the alternative exemplary arrangement. Further it will be appreciated that the insertion tool 1000, 1000′ may be configured for use with other implants or bone anchors.

Referring to FIGS. 10a to 10c, features of the handle assembly of the insertion tool 1000, 1000′ and operation of the handle assembly is described in further detail.

Referring to FIG. 10a the handle is shown in a first engaged operational position at which the sutures are engaged in the insertion tool. Referring to FIGS. 10b and 10c, the handle is shown in a second released operational position at which the insertion tool is released and the sutures are released from the handle. In the first position, the actuator 1013 is located at a first upper position of the range of movement of the actuator where it is located spaced apart a distance d1 from the upper proximal portion 1005 of the body 1011. The actuator 1013 is configured such that when the user exerts a force on the actuator 1013, it is moveable downwardly to the second lower position of the range of movement of the actuator. In the exemplary arrangement of FIGS. 10b and 10c, at the second lower position, the lower surface of the actuator 1013 is brought into contact with the upper surface of the cap portion 1014 of the handle assembly 1010.

The external wall 1015 of the handle assembly comprises first and second channels 1018. The first and second channels are formed recessed relative to the external wall and are open to the external facing surface. The channels extend in the longitudinal direction from first and second openings 1023 to the proximal end 1007 of the handle portion 1010. From the handle portion the sutures 610 extend into the sleeve 1050 parallel to the shaft 1030.

Each of the first and second openings 1023 comprises a cover 1021 connected to the handle at a hinge 1022. The cover is also referred to in the specification as a door flap 2021. Each cover or door flap is attached to the handle at a hinge 2022. The hinges are oriented in the generally horizontal direction at the upper side to the cover providing an attachment of the cover to the handle. Each is moveable between a closed position at which the cover rests in proximity to the wall 1015 and an open position at which the cover is opened at an angle relative to the wall. The movement of the covers 1021 is controlled by the actuator 1013. The covers 1021 are coupled to the actuator 1013 via levers 1026 and/or needles 1027. The covers 1021 are positioned such that the hinge connection is located to the proximal side of the covers 1021 and opening 1023. When the covers are opened, they open such that the lower edge of the cover is displaced upwardly and outwardly relative to the handle, the cover remains attached at the upper side edge to the handle. The covers 1021 are configured to protect the user's hand from the needles located internally within the housing 1012 of the handle assembly 1010.

The sutures 610 which extend proximally from their distal-most position in the bone anchor 69 are located in the channels 1018 and connected to the needles 1027. The sutures 610 are connected to the needles internally in the housing 1012. The sutures 610 extend from the upper ends of the channels 1018 through openings 1023 to the needles 1027 located internally in the housing.

Referring to FIGS. 10b and 10c when the user exerts a force on the actuator 1013, it moves downwardly to the second lower position of the range of movement of the actuator. At the second lower position, the lower surface of the actuator 1013 is brought into contact with the upper surface of the cap portion 1014 of the handle assembly 1010.

The movement of the actuator 1013 to the second lower position causes movement of a hinge element 1025 and levers 1026 connected to the actuator and located internally in the housing 1012. The overall effect is to release the sutures 610 from the needles 1027 and to release the sutures 610 from their position internally in the housing 1012 via opening 1023.

When actuator 1013 is moved to the second lower position, the levers 1026 connected thereto in turn move, resulting in exertion of forces in directions axially outwardly relative to the central vertical axis. Forces are exerted firstly on the needles 1027 and secondly on the covers 1021. The upper proximal ends of the sutures 610 are released and the sutures are released from the insertion tool.

Advantageously, the actuator is operable to release the sutures and to allow the user to release the insertion tool from the bone anchor and the surgical site. The actuator 1013 which as described above is provided in this arrangement as a push button allows for single handed operation and for single handed release of the sutures from the needles.

The insertion tool and bone anchor may be provided as a preloaded kit. The insertion tool and bone anchor may be provided as a single use preloaded kit. The insertion tool may be provided comprising each of the three portions the handle portion as described above, the shaft and the sleeve. The insertion tools 1000, 1000′ and bone anchors 39, 49, of the arrangements of the specification are configured for use in methods for application or insertion of a bone anchor at a surgical device. The method allows for location of the bone anchor and sutures as described. The method further allows for the provision of a fluid for example a bone cement or other adhesive at the surgical site in proximity to the bone anchor. The kit allows for control of the location and positioning of sutures at the implant and at the surgical site, as required.

Overall the specification provides arrangements of an insertion tool 1000, 1000′ for inserting a bone anchor as claimed in any preceding claim into bone at a surgical site, the bone anchor comprising a distal tunnel and first and second longitudinally extending passageways located on opposing sides of the bone anchor, the insertion tool comprising a proximal handle 1010 and an elongated shaft 1030 coupled at a first proximal end to the handle and configured for coupling at a second distal end to the bone anchor for inserting the bone anchor, the bone anchor is configured to be inserted with a suture located in the bone anchor, the suture extending through the first and second longitudinal passageways and tunnel of the bone anchor.

The insertion tool comprises a bone anchor engagement means 1030, 1060 and suture engagement means 1018, 1027, 1023, 1022—and provides the user with means to control both. The bone anchor engagement means comprising a distal head 1060 of the shaft 1030 configured to be received in a corresponding socket 615 of the bone anchor, the insertion tool configured such that a user actuation of the insertion tool provides for driving of the bone anchor into the bone. The bone anchor is driven by the movement of the shaft. The suture engagement means comprising first and second needles located in the handle, first and second ends of the suture extends in the proximal direction from the bone anchor for engagement with the insertion tool at said needles. The suture engagement means further includes a suture retainer formed on the external surface of the handle. In the exemplary arrangement this suture retainer is provided as channels 1018. In addition, the sutures are extended though the sleeve from the bone anchor to the handle. As described the insertion tool also comprises means to allow for the controlled introduction of a fluid to the surgical site.

Whilst the above explains the present disclosure by reference to rotator cuff surgery, the present disclosure is not limited to this use. The bone anchor can be used in any situation where a strong anchoring point to bone is required. In particular, the bone anchor may be used in surgical operations at the knee, ankle, elbow, wrist, or hip.

1. A bone anchor for rotator cuff repair, comprising:
a solid cylinder extending in a proximal to distal direction and defining a longitudinal axis;
a thread on the solid cylinder encircling the solid cylinder at least twice;
a connection, at a proximal end of the solid cylinder, for connecting to a tool to drive the thread into bone;
an unthreaded endpiece, extending from a distal end of the solid cylinder, having a distal end; and
a tunnel across the unthreaded endpiece,
wherein the thread is configured to define at least two straight longitudinal passageways from the distal end of the solid cylinder to the proximal end of the solid cylinder.

2. The bone anchor of claim 1, wherein the tunnel is a straight tunnel perpendicular to the longitudinal axis.

3. The bone anchor of claim 2, wherein the straight tunnel defines a circular cross section that is centred on the longitudinal axis.

4. The bone anchor of claim 1, wherein the at least two straight longitudinal passageways are equally spaced around a circumference of the solid cylinder.

5. The bone anchor of claim 1, wherein a depth of the thread, measured from the solid cylinder varies, along the thread.

6. The bone anchor of claim 1, wherein a proximal side of a thread profile has at least 30 degrees less inclination from the longitudinal axis than a distal side of the thread profile.

7. The bone anchor of claim 6, wherein a protrusion of the thread from the solid cylinder decreases by at least 0.5 mm from the proximal end of the thread to the distal end of the thread.

8. The bone anchor of claim 6, wherein a protrusion of the thread at the distal end of the thread is either less than 0.5 millimeters; or is 0.3 mm or less.

9. The bone anchor of claim 8, wherein a difference in inclination, between the proximal side of the thread profile and the distal side of the thread profile from the longitudinal axis, increases from the proximal end of the thread to the distal end of the thread.

10. The bone anchor of claim 1, wherein the unthreaded endpiece has been plasma processed or anodized.

11. The bone anchor of claim 1, wherein the unthreaded endpiece defines a cylindrically symmetric shape comprising:
a cylinder; and
a rounded end at a distal end of the cylinder,
the rounded end defining a radius of between 1 mm and 5 mm.

12. The bone anchor of claim 11, wherein the end radius is at least as large as the radius of the solid cylinder or is the radius of the solid cylinder.

13. The bone anchor of claim 1, wherein at least the unthreaded endpiece comprises Grade 5 Titanium.

14. The bone anchor of claim 1, wherein the bone anchor has an overall longitudinal length of between 5 mm and 50 mm, or between 10 mm and 30 mm, or is 20 mm.

15. The bone anchor of claim 1, wherein the at least two straight longitudinal passageways and tunnel are configured to receive and retain one or more sutures at the bone anchor as the bone anchor is inserted at a surgical site.

16. A kit, for rotator cuff repair surgery, comprising:
a bone anchor according to claim 1; and
a bone adhesive.

17. An insertion tool for inserting a bone anchor as claimed in claim into bone at a surgical site, the bone anchor comprising a distal tunnel and first and second longitudinally extending passageways located on opposing sides of the bone anchor, the insertion tool comprising:
a proximal handle; and
an elongated shaft coupled at a first proximal end to the proximal handle and configured for coupling at a second distal end to the bone anchor for inserting the bone anchor, the bone anchor is configured to be inserted with a suture located in the bone anchor, the suture extending through the first and second longitudinal passageways and tunnel of the bone anchor;
wherein the insertion tool comprises a bone anchor engagement means and suture engagement means;
the bone anchor engagement means comprising a distal head configured to be received in a corresponding socket of the bone anchor, the insertion tool configured such that a user actuation of the insertion tool provides for driving of the bone anchor into the bone; and
the suture engagement means comprising first and second needles located in the proximal handle, wherein first and second ends of the suture extend in a proximal direction from the bone anchor for engagement with the insertion tool at the first and second needles.

18. The insertion tool of claim 17, further comprising a release actuator configured to release the suture from the insertion tool.

19. The insertion tool of claim 17, wherein the proximal handle comprises a housing and the first and second needles are contained within the housing.

20. The insertion tool of claim 18, wherein a housing comprises first and second externally located longitudinal channels and first and second openings, wherein the suture is receivable in the longitudinal channels and threaded therefrom through the first and second openings for engagement with the first and second needles.

21. The insertion tool of claim 20, wherein each opening comprises a cover configured to open to release the suture and configured to define a protective covering for the needles to protect hands of a user from the needles.

22. The insertion tool of claim 17 further comprising:
a sleeve, which in use is located between the proximal handle and the bone anchor and the surgical site,
the sleeve having a cylindrical wall comprising an internal channel, the insertion tool configured such that the elongated shaft is located extending through the internal channel of the sleeve and movable relative to the sleeve;
and further wherein the sleeve is configured to receive the suture extending proximally from the bone anchor in the direction of the proximal handle.

23. The insertion tool of claim 22, further comprising a fluid input which comprises a syringe dock for receiving a syringe and a fluid entry port, the fluid entry port formed as an aperture that extends through the cylindrical wall to allow for delivery of a fluid to the surgical site via the sleeve.

24. The insertion tool of claim 23, further comprising a stop or lock located at the sleeve and that is operable to allow for the shaft to be retained proximally of the fluid entry port, to allow for a fluid to be introduced into the sleeve, and to allow for release of the shaft when the fluid has been introduced.

25. A sleeve for use with an insertion tool for inserting an implant, the sleeve having a cylindrical wall defining an internal channel or lumen for receiving a tool or other component used in surgery, and comprising a fluid input comprises a syringe dock for receiving a syringe and a fluid entry port, the fluid entry port formed as an aperture that extends through the cylindrical wall to allow for delivery of a fluid to a surgical site via the sleeve.

26. The sleeve of claim 25, further comprising a stop or lock located at the sleeve and that is operable to allow for the tool to be retained proximally of the fluid entry port, to allow for a fluid to be introduced into the sleeve, and to allow for release of a shaft when the fluid has been introduced.

27. (canceled)
